(19) United States
(12) Patent Application Publication
(10) Pub. No.: US 2006/0051366 A1
(43) Pub. Date: Mar. 9, 2006

(54) Use of soluble CD26 as inhibitor of angiogenesis and inflammation

(76) Inventor: Chiwen Chang, Cambridge (GB)

Correspondence Address: HELLER EHRMAN LLP, 275 MIDDLEFIELD ROAD, MENLO PARK, CA, 94025-3506, US

(21) Appl. No.: 11/208,288

(22) Filed: Aug. 18, 2005

Related U.S. Application Data
(60) Provisional application No. 60/605,013, filed on Aug. 26, 2004.

(51) Int. Cl. A61K 39/00

(52) U.S. Cl. 424/192.1

(57) ABSTRACT

The present invention relates to soluble CD26 or a biologically active variant of the soluble CD26 that effectively inhibits angiogenesis and inflammation in a mammalian subject, pharmaceutical compositions comprising the soluble CD26 and the use of soluble CD26 in methods of treatment.

### RELATED APPLICATIONS

The present application claims the benefit of priority from commonly assigned co-pending U.S. Provisional Application Ser. No. 60/605,013 filed Aug. 26, 2004. This application is fully incorporated herein by reference for all purposes.

### BACKGROUND OF THE INVENTION

1. Field of the Invention

The present invention relates generally to the treatment of diseases or disorders which are dependent on angiogenesis and inflammation. In particular, the present invention relates to the methods of inhibiting angiogenesis by administering an effective amount of soluble CD26, wherein soluble CD26 is capable of inhibiting VEGF activity and IL-2 activity.

2. Description of the Related Art

Angiogenesis

Angiogenesis is the formation of new blood vessels by sprouting from pre-existing ones. (Weinstate-Saslow, The FASEB Journal 8: 402-407, 1994; Folkman et al., Science 235: 442-447, 1987). The generation of new blood vessels involves a multistep process, which includes the migration of vascular endothelial cells into tissue, followed by the condensation of such endothelial cells into vessels. Angiogenesis may be induced by an angiogenic agent or be the result of a natural condition. The process is essential to a variety of normal body activities, such as embryo implantation; embryogenesis and development; and wound healing. The process involves a complex interplay of molecules that stimulate and molecules that inhibit the growth and migration of endothelial cells, the primary cells of the capillary blood vessels. (Folkman and Shing, J. Biol. Chem., 267 (16): 10931-34, 1989; Folkman and Klagsbrun, Science, 235, 442-47, 1987).

Several angiogenic agents have been identified. (Hanahan and Folkman, Cell, 86 (3): 353-364, 1996). For example, a number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF): and platelet derived growth factor (PDGF) (Ferrara and Davis-Smyth, Endocr Rev. 18 (1): 4-25, 1997). VEGF is believed to be a central mediator of angiogenesis. Antibodies directed against VEGF have been shown to suppress tumor growth in vivo and decrease the density of blood vessels in experimental tumors (Kim et al., Nature 362: 841-844, 1993), indicating that VEGF antagonists could have therapeutic applications as inhibitors of tumor-induced angiogenesis.

Normal angiogenic activity is low in healthy adults and limited to certain organs such as the uterus during pregnancy or intensely exercising skeletal muscle. However, its activity increases during injury and in diseases such as cancer, retinopathy, or arthritis, where it contributes to pathological changes. Therefore, angiogenesis can have both the beneficial effects such as facilitating wound healing, and detrimental effects by causing inflammatory diseases such as, for example, rheumatoid arthritis, macular degeneration, psoriasis, and diabetic retinopathy.

Furthermore, it has been shown that angiogenesis is essential for the growth of solid tumors and for tumor metastasis (Bouck et al., Adv Cancer Res.; 69: 135-74, 1996; Yancopoulos et al., Nature 407 (6801): 242-8, 2000). Tumor-induced angiogenesis is initiated by growth factors and cytokines that are released from the tumor or from inflammatory cell infiltrates (Brown et al., Am. J. Path. 143: 1255, 1993; Brown et al., Human Path. 26: 86, 1995; Leek et al., J. Leukocyte Biol. 56: 423, 1994; Hatva et al., Am. J. Pathol. 146: 368, 1995; and Plate et al., Nature 359: 845, 1992). Growth factors and cytokines which are expressed by tumor cells stimulate angiogenesis in a number of animal models including the chick chorioallantoic membrane model, the corneal pocket angiogenesis model, and models involving spontaneous and xenotransplanted tumor growth (Brooks et al., Cell 79: 1157, 1994; Brooks et al., Science 264: 569, 1994; Brooks et al., J. Clin. Invest. 96: 1815, 1995; and Friedlander et al., Science 27: 1500, 1995). Accordingly, tumor-associated angiogenesis is a potential target for therapies that inhibit tumor proliferation, invasion, and metastasis since angiogenesis has been implicated not only in the growth of tumors but also in their metastasis (Liotta et al., 1991, Cell 64: 327; Weinstat-Saslow et al., FASEB J 8: 401, 1994; Blood et al., Biochim. Biophys. Acta 1032: 89, 1990; Folkman, Semin. Cancer Biol. 3: 65, 1992; and Weidner et al., N. Engl. J. Med. 324: 1, 1991).

It is now well recognized that angiogenesis is involved in a variety of diseases or disorders and that such diseases or conditions can be treated by administration of angiogenesis inhibitors. Examples of pathological conditions involving angiogenesis include but are not limited to, macular degeneration; ocular neovascular glaucoma; diabetic retinopathy; corneal graft rejection; vitamin A deficiency; Sjorgen's disease; acne rosacea; mycobacterium infections; bacterial and fungal ulcers; Herpes simplex infections; systemic lupus; rheumatoid arthritis; osteoarthritis; psoriasis; chronic inflammatory diseases (e.g., ulcerative colitis, Crohn's disease); hereditary diseases such as Osler-Weber Rendu disease and haemorrhagic teleangiectasia.

In an attempt to treat these diseases or conditions, many angiogenesis inhibitors have been discovered. Examples include endostatin (O'Reilly et al., 1997, Cell 88: 277); angiostatin (O'Reilly et al., 1994, Cell 79: 315); the peptide CNGRCVSGCAGRC (Arap et al., 1998, Science 279: 377); the cyclic peptide RGDfV (Friedlander et al., 1995, Science 270: 1500); and the monoclonal antibodies LM609 and P1F6 (Friedlander et al., 1995, Science 270: 1500).

CD26

Human CD26, also known as a dipeptidyl peptidase IV (DPPIV), is a 240 kDa homodimeric type II membrane glycoprotein composed of two 120 kDa subunits. (Mentlein, R., International Review of Cytology, 235: 165-213, 2004). It is a cell surface glycoprotein with various functional properties, which includes modulating the activity of various biologically important peptides. (Dang et al., Histol. Histopathol., 17: 1213-1226, 2002). It is expressed on a variety of cell types, particularly melanocytes, epithelial cells, endothelial cells and lymphocytes. In addition to the membrane-bound form, soluble CD26 (“sCD26”), which lacks the first 38 residues, has been postulated as a cleaved product from the membrane CD26. (Iwaki-Egawa et al., J. Biochem. (Tokyo) 124; 428-433 (1998)). On the other hand, a human placental soluble CD26, which is a cleaved product from a membrane CD26 from a placenta, lacks only the first 28 residues (See FIG. 8). Therefore, a human placental sCD26 has an additional 10 amino acid residues at its N-terminus. FIG. 4 illustrates how a placental homodimeric soluble CD26 may be generated from a membrane-bound homodimeric CD26. The transmembrane region of a membrane CD26 is underlined and the cleavage site is indicated by an arrow in FIG. 4.

The amino acid sequences of native CD26 are known in the art. The full-length amino acid sequence of human membrane CD26 is shown in FIG. 6 and the amino acid sequence of murine membrane CD26 is shown in FIG. 10.

Soluble CD26 can cleave NH₂-terminal dipeptides from polypeptides with either L-proline or L-alanine at the penultimate position (Fleischer, Immunol Today 15; 180-184 (1994)). Many biologically active polypeptides have this sequence. For example, a proline residue is present at the penultimate position in many cytokines, such as IL-1β, IL-2, IL-6, and G-CSF. (Ansorge et al., Biomed Biochim Acta 50; 799-807 (1991)).

Studies have shown that sCD26 has many physiological roles, including a role in immune regulation as a structure capable of transmitting T cell activation signals and a role as a regulator of biological processes through its cleavage of biological factors. In addition, it appears that sCD26 may be involved in the development of certain human cancers. (Dang et al., Histol. Histopathol., 17: 1213-1226, 2002). It has been shown that the levels of sCD26 are altered in adults with certain diseases or conditions, such as cancer, when compared to those levels detected in healthy adults. (Cordero et al., British Journal of Cancer, 83 (9); 1139-1146 (2000)).

IL-2

Activation of a T cell is a complex process involving various secreted interleukins, which acts as local chemical mediators. Activation is thought to begin when the T cell, by unknown means, is stimulated by the antigen-presenting cell to secrete one or more interleukins. Interleukin 2 (IL-2) is a protein produced by T-lymphocytes that have been activated by an antigen. IL-2 stimulates other lymphocytes to activate and differentiate. IL-2 is a central cytokine required for the activation of T, B and natural-killer (NK) cells. (Tenbrock et al., Int Rev Immunol., 23 (3-4); 333-345, 2004).

Human IL-2 is a protein of 133 amino acids (15.4 kDa) with a slightly basic pI that does not display sequence homology to any other factors. Murine and human IL-2 display a homology of approximately 65%. IL-2 is synthesized as a precursor protein of 153 amino acids with the first 20 amino-terminal amino acids functioning as a hydrophobic secretory signal sequence. The protein contains a single disulfide bond at positions Cys58 and Cys105, which is essential for biological activity.

It is well known in the art that IL-2 is a pro-inflammation cytokine in the human immune system. There are numerous examples of the pathological role IL-2 plays in the inflammatory and immune diseases. For example, the level of IL-2 production is altered in patients suffering from the multiple sclerosis and reduced in patients suffering from systemic lupus erythematosus. (Dejica D., Rorum Arch Microbiol Immunol., 60 (3): 183-201, 2001; Herndon et al., Clin. Immunol., 103 (2), 145-53, 2002; Tenbrock et al., Int Rev Immunol., 23 (3-4): 333-45, 2004).

Therefore, development of a molecule of which would alter the IL-2 activity would be useful in the treatment of autoimmune and inflammatory disorders.

### SUMMARY OF THE INVENTION

We have now found that a soluble CD26 is capable of inhibiting VEGF activity and IL-2 activity.

We have determined that angiogenesis induced by the proliferation of endothelial cells is inhibited by the effect of soluble CD26, or a biologically active variant of soluble CD26 on the activity of VEGF. The VEGF activity is a prerequisite for the progression of an angiogenesis-dependent condition, such as the continued growth of a vascular tumor and of its metastatic lesions. The administration of soluble CD26 or a biologically active variant of soluble CD26 to a patient therefore provides a way of preventing angiogenesis, thereby arresting the progression of an angiogenesis-dependent disease or condition. Preferably the soluble CD26 is human CD26. More preferably, the soluble CD26 is placental soluble CD26.

We have also determined that the proliferation of endothelial cells is inhibited by the effect of soluble CD26 or a biologically active variant of soluble CD26 on the activity of VEGF. The VEGF activity is a prerequisite for the progression of an angiogenesis-dependent condition, such as the continued growth of a vascular tumor and of its metastatic lesions. The administration of soluble CD26 or a biologically active variant of soluble CD26 to a patient therefore provides a way of preventing angiogenesis, thereby arresting the progression of an angiogenesis-dependent disease or condition. Preferably the soluble CD26 is human CD26. More preferably, the soluble CD26 is placental soluble CD26.

The present invention is also based on the finding that the soluble CD26 or a biologically active variant of placental soluble CD26 is capable of inhibiting angiogenesis and inflammation in a mammalian subject.

We have found that cleavage of the VEGF at the CD26 recognition motif sequence inactivates VEGF. For example, in one embodiment, cleaving after the proline residue at amino acid position 2 of the mature VEGF inactivates the VEGF. (See FIG. 12(A)). Accordingly, removal of the alanine-proline residues at positions 1 and 2 of the mature VEGF inactivates VEGF.

One embodiment of the present invention is to provide a method of inhibiting VEGF activity, comprising contacting VEGF with an effective amount of soluble CD26 or a biologically active variant of soluble CD26. Preferably the soluble CD26 is human CD26. More preferably, the soluble CD26 is placental soluble CD26.

Yet another embodiment of the present invention is to provide a method of inhibiting angiogenesis in a mammalian subject, comprising administering to said subject a therapeutically effective amount of soluble CD26 or a biologically active variant of soluble CD26. In a preferred embodiment, the mammalian subject is human. Preferably the soluble CD26 is human CD26. More preferably, the soluble CD26 is placental soluble CD26.

In one aspect, the present invention concerns a method of treating a disease or a condition associated with angiogenesis in a mammalian subject, comprising administering to said subject a therapeutically effective amount of soluble CD26 or a biologically active variant of soluble CD26. In a preferred embodiment, the mammalian subject is human. In one embodiment, the disease is cancer. In a particular embodiment, the cancer is lymphoma. In another embodiment, the cancer is leukemia.

In another embodiment, the invention concerns a pharmaceutical composition comprising soluble CD26 or a biologically active variant of soluble CD26, or a pharmaceutically acceptable salt thereof, together with at least one pharmaceutically acceptable carrier. Preferably the soluble CD26 is human CD26. More preferably, the soluble CD26 is placental soluble CD26.

In yet another embodiment, the invention concerns a method for inhibiting IL-2 activity in a human subject, comprising administering an effective amount of soluble CD26 or a biologically active variant of soluble CD26, or a pharmaceutically acceptable salt thereof. In a preferred embodiment, the mammalian subject is human. Preferably the soluble CD26 is human CD26. More preferably, the soluble CD26 is placental soluble CD26.

In one aspect of the invention, cleavage of IL-2 at the CD26 recognition motif sequence inactivates IL-2. For example, in one embodiment, cleaving after the proline residue at amino acid position 2 of the mature IL-2 inactivates the IL-2. (See FIG. 12(B)). Accordingly, removal of the alanine-proline residues at positions 1 and 2 of the mature IL-2 inactivates IL-2.

Another embodiment of the present invention is to provide a method of inhibiting inflammation in a mammalian subject, comprising administering to said subject a therapeutically effective amount of soluble CD26 or a biologically active variant of soluble CD26.

In one aspect, the invention concerns a method of treating an inflammatory, immune or autoimmune disease in a mammalian subject, comprising administering to said subject a therapeutically effective amount of soluble CD26 or a biologically active variant of soluble CD26.

In one embodiment, the soluble CD26 or a biologically active variant of soluble CD26, of the present invention may be administered permucosally, orally, enterally, percutaneously, subcutaneously, transdermally, intravenously, by aspiration, by suppository, by instillation, endoscopically, intratracheally, intralesionally, intratumorally, intramuscularly or via mucous membranes such as in a nasal spray.

In a further aspect, the invention concerns an antibody that binds to placental soluble CD26 or a biologically active variant of placental soluble CD26. In one embodiment, the placental soluble CD26 or the biologically active variant of placental soluble CD26 is a monomer. In another aspect, the placental soluble CD26 or the biologically active variant of placental soluble CD26 is a dimer. In yet another embodiment, the antibody is an antagonist antibody. In a further embodiment, the antibody is a monoclonal antibody. In another embodiment, the antibody is a humanized antibody. In yet another embodiment, the antibody is an antibody fragment. In another embodiment, the antibody is labeled.

In a particular embodiment, the soluble CD26 of the present invention is a human placental soluble CD26, which lacks the first 28 N-terminal residues from its membrane CD26.

In another embodiment the invention is directed to the use of soluble CD26 for the manufacture of a pharmaceutical composition for the treatment of a disease or a condition associated with angiogenesis in a mammalian subject. The invention is directed to the use of soluble CD26 for the manufacture of a pharmaceutical composition wherein the patient has been diagnosed with or is at risk of developing a condition selected from cancer, macular degeneration ocular neovascular glaucoma; diabetic retinopathy; corneal graft rejection; vitamin A deficiency; Sjorgen's disease; acne rosacea; mycobacterium infections; bacterial and fungal ulcers; Herpes simplex infections; systemic lupus; rheumatoid arthritis; osteoarthritis; psoriasis; chronic inflammatory diseases (e.g., ulcerative colitis, Crohn's disease); hereditary diseases such as Osler-Weber Rendu disease and haemorrhagic teleangiectasia.

In another embodiment the invention is directed to the use of soluble CD26 for the manufacture of a pharmaceutical composition for the treatment of a disease or a condition associated with inflammatory, immune or autoimmune disease in a mammalian subject. The invention is directed to the use of soluble CD26 for the manufacture of a pharmaceutical composition wherein the patient has been diagnosed with or is at risk of developing a condition selected from rheumatoid arthritis (RA), psoriasis, rheumatoid spondylitis, gouty arthritis; autoimmune diabetes, autoimmune hepatitis; multiple sclerosis (MS), asthma, systemic lupus erythematosus, Wegener's granulomatosis (WG), kidney inflammation, lupus nephritis, chronic pulmonary inflammatory disease, inflammatory bowel disease (IBD), Alzheimer's disease and ocular allergy.

### BRIEF DESCRIPTION OF THE DRAWINGS

FIG. 1 shows co-culture of decidual leukocytes with human placental trophoblasts.

FIG. 2 shows the inhibition of VEGF detection by human placental sCD26 and the blocking of the inhibition of VEGF detection by the anti-placental sCD26 antibody.

FIG. 3 shows the immunoprecipitation of human placental sCD26 by anti-placental sCD26 monoclonal antibody from the supernatant of cultured trophoblast cells.

FIG. 4 shows schematic diagram of a human placental membrane and soluble CD26 and the cleavage site after 28 amino acids in the N-terminus of membrane CD26. The amino acid residues of the transmembrane region are underlined.

FIG. 5 shows the nucleic acid sequence encoding the human membrane CD26 (SEQ ID NO:1).

FIG. 6 shows the amino acid sequence of the human membrane CD26 (SEQ ID NO:2).

FIG. 7 shows the nucleic acid sequence encoding the human placental soluble CD26 (SEQ ID NO:3).

FIG. 8 shows the amino acid sequence of the human placental soluble CD26 (SEQ ID NO:4).

FIG. 9 shows nucleic acid sequence encoding the murine membrane CD26 (SEQ ID NO:5).

FIG. 10 shows the amino acid sequence of the murine membrane CD26 (SEQ ID NO:6).

FIG. 11 shows the effect of human placental soluble CD26 on the proliferation of HUVEC by VEGF stimulation.

FIG. 12(A) shows the first 15 N-terminal amino acid residues of mature, secreted VEGF-A. The soluble CD26 recognition motif sequence is underlined.

FIG. 12(B) shows the first 15 N-terminal amino acid residues of mature, secreted IL-2. The soluble CD26 recognition motif sequence is underlined.

FIG. 13 shows the pre-processed amino acid sequence of human VEGF-A.

FIG. 14 shows the pre-processed amino acid sequence of human IL-2.

FIG. 15 shows the inhibition of IL-2 activity by human placental soluble CD26.

FIG. 16 is a chart showing the effect of placental sCD26 in human blood on VEGF activity as measured by ELISA. The vertical axis is the OD₄₅₀. Lane 1 is VEGF alone, lane 2 is VEGF and trophoblast supernatant, lane 3 is VEGF and pregnant women's serum, lane 4 is VEGF and pregnant women's serum and anti-placental CD26 antibody CH15; lane 5 is VEGF and male serum; lane 6 is VEGF and male serum and antibody CH15; lane 7 is VEGF and non-pregnant women's serum, lane 8 is VEGF and non-pregnant women's serum and antibody CH15.

FIG. 17 is a chart showing the effect of pregnant women's blood serum on VEGF activity as measured by ELISA. Lane 1 is VEGF alone; lane 2 is VEGF and trophoblast supernatant, lane 3 is VEGF and pregnant women's serum from the second trimester; lane 4 is VEGF and pregnant women's serum from the third trimester and lane 5 is VEGF and pregnant women's serum from 5 month's after delivery.

FIG. 18 is a chart showing the level of VEGF digestion by human blood serum from males, non-pregnant females and pregnant females.

### DETAILED DESCRIPTION OF THE PREFERRED EMBODIMENT

### A. DEFINITIONS

Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “CD26”, “protease dipeptidyl peptidase IV”, “dipeptidyl peptidase IV”, “DPPIV” and “DP IV” are used interchangeably and are used herein in the broadest sense, and includes all naturally occurring CD26 molecules of any animal species, including the homodimer and subunits thereof, and all naturally occurring mutant and variant forms thereof, and its functional derivatives, such as amino acid sequence variants.

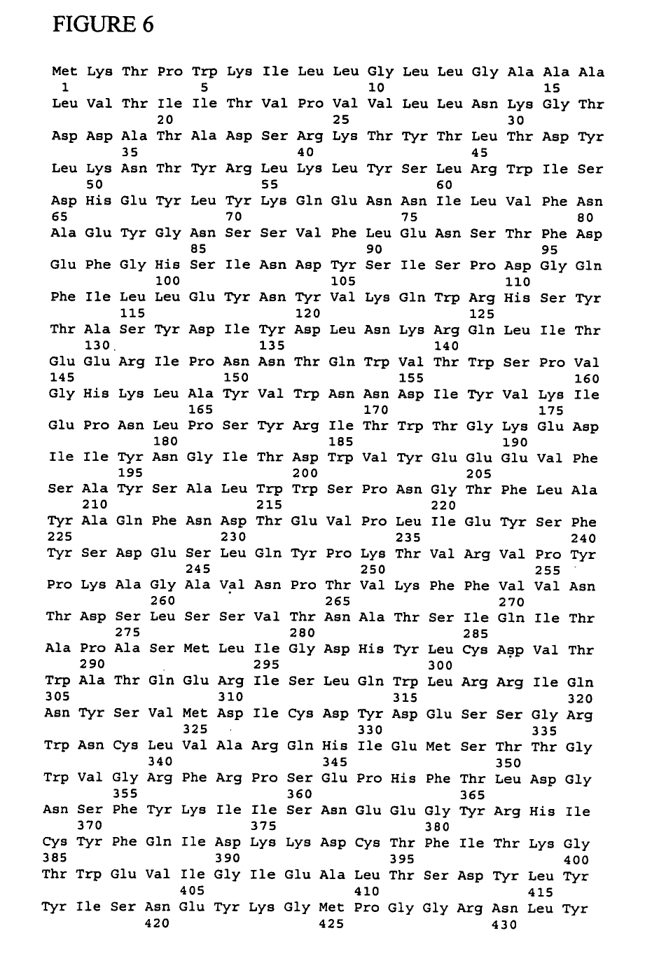

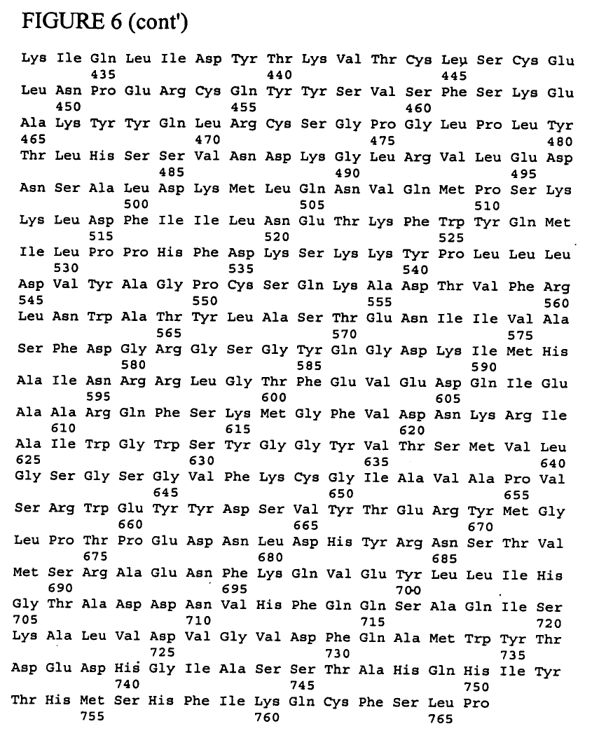

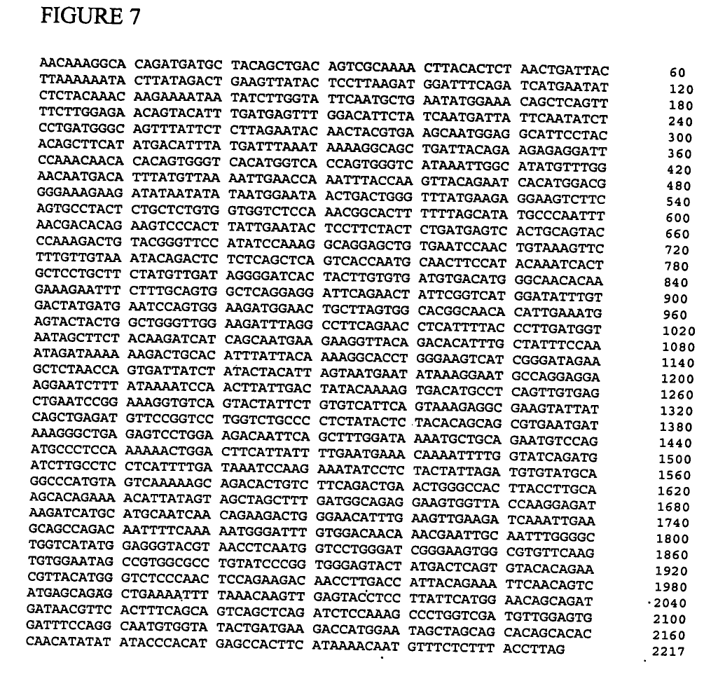

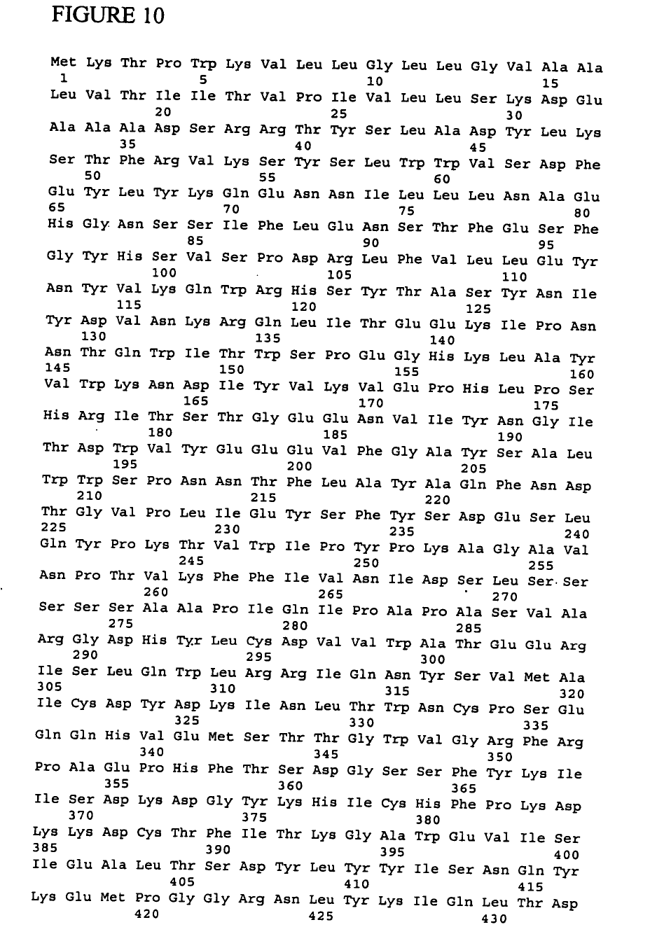

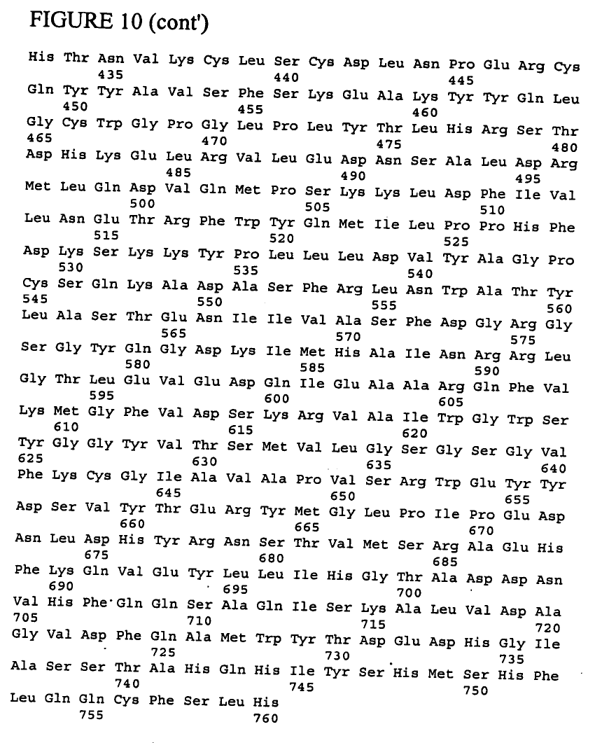

The term “membrane CD26” is used herein in the broadest sense, and includes all full-length naturally occurring mature CD26 molecules of any mammal species, including the homodimer and subunits thereof, and all naturally occurring mutant and variant forms thereof, and its functional derivatives, such as amino acid sequence variants. Membrane CD26 is preferably about 766 amino acids in length. Preferably the membrane CD26 is human membrane CD26. Preferably, the membrane CD26 is the amino acid sequence set forth in FIG. 6 or FIG. 10.

As used herein, the expressions “soluble CD26” and “sCD26” are used interchangeably and all such designations refer to a soluble form of the cell-surface glycoprotein CD26 expressed on a variety of mammalian cell types. Preferably the soluble CD26 is a human soluble CD26. Preferably, the soluble CD26 comprises a truncated or spliced version of membrane CD26 wherein a portion of the N-terminus of membrane CD26 has been deleted. Preferably soluble CD26 comprises at least about 728 contiguous amino acids of membrane CD26 wherein the N-terminus of membrane CD26 has been deleted. Preferably soluble CD26 comprises at least the sequence set forth in FIG. 6 from amino acid 39 to amino acid 766. Soluble CD26 includes placental soluble CD26.

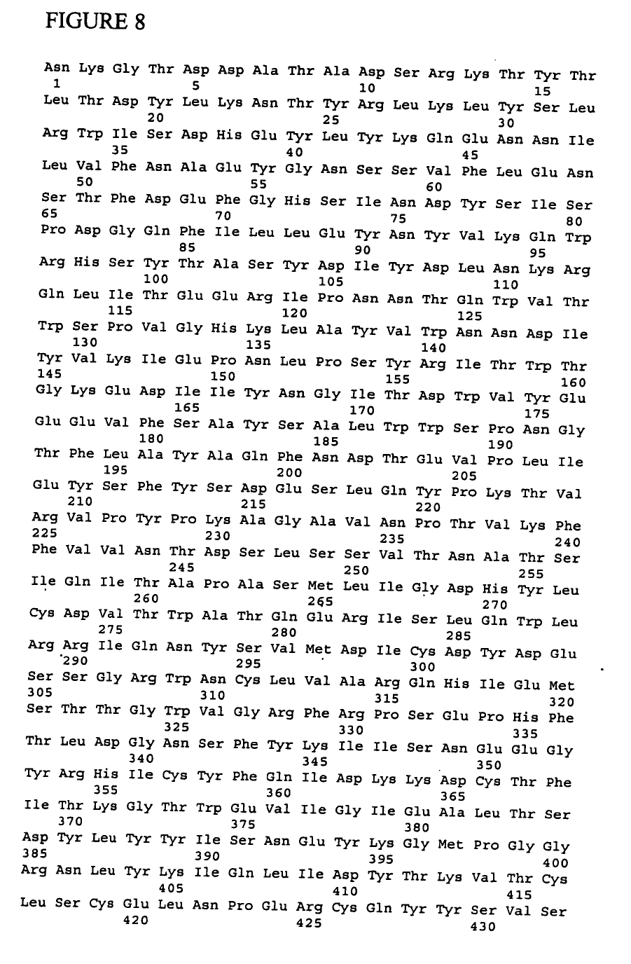

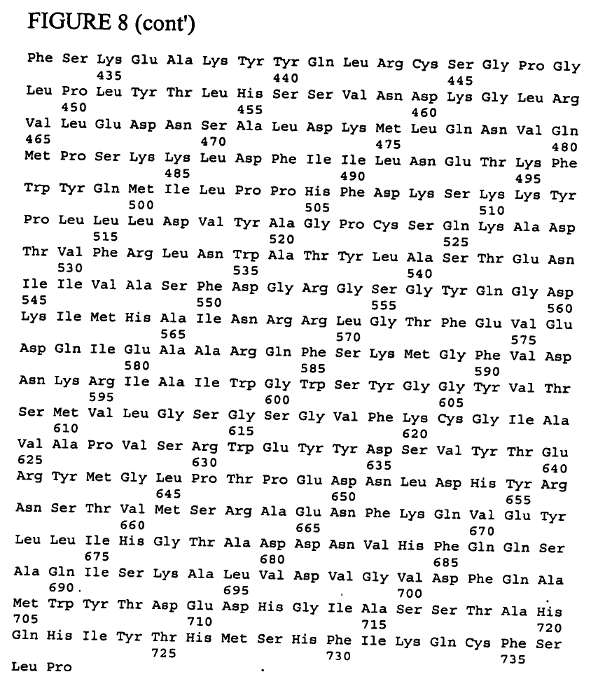

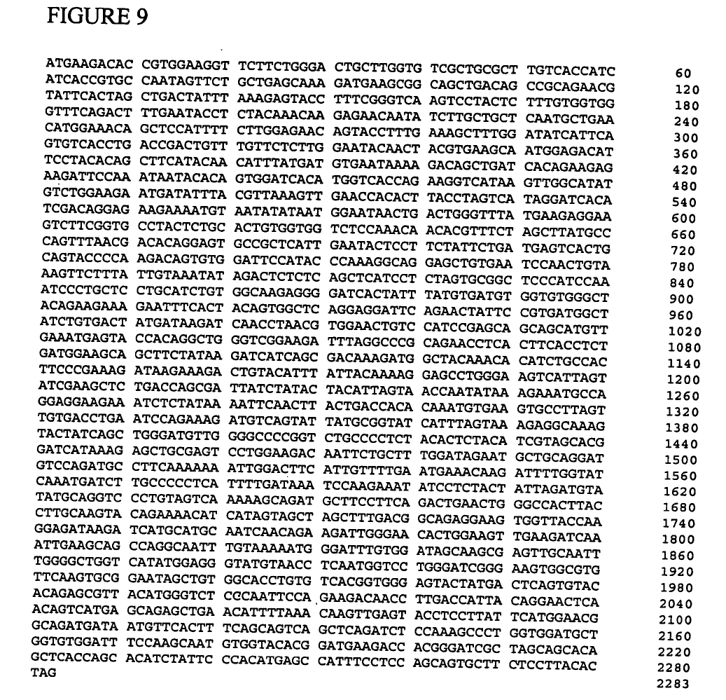

The expressions “placental soluble CD26” and “placental sCD26”, as used herein, are used interchangeably and all such designations refer to a soluble CD26 comprising at least about 10 additional amino acids at the N-terminus. Preferably placental soluble CD26 is mammalian placental soluble CD26, more preferably it is human placental soluble D26. The terms “placental soluble CD26” or “placental sCD26” does not mean that the protein must be obtained from placenta tissue. This splice variant was originally identified in the placenta tissue, but could be generated from any membrane CD26 by removing the first 28 N-terminal amino acid residues. Preferably placental soluble CD26 comprises at least about 738 contiguous amino acids of membrane CD26 wherein the N-terminus of membrane CD26 has been deleted. Preferably placental soluble CD26 comprises at least the sequence set forth in FIG. 8.

The soluble CD26 encompassed by the present invention includes analogues and variants thereof having the biological activity of native soluble CD26. The soluble CD26 includes all naturally occurring mutant and variant forms thereof, and its functional derivatives, such as amino acid sequence variants. The amino acid sequence variants of soluble CD26, includes but is not limited to the populations of variants generated using gene shuffling. The placental soluble CD26 encompassed by the present invention includes analogues and variants thereof having the biological activity of native placental soluble CD26. The placental soluble CD26 includes all naturally occurring mutant and variant forms thereof, and its functional derivatives, such as amino acid sequence variants. The amino acid sequence variants of placental soluble CD26, include but are not limited to the populations of variants generated using gene shuffling. Gene shuffling or DNA shuffling is a method that generates diversity by recombination as described, for example, in Stemmer, Proc. Natl. Acad. Sci. USA 91: 10747-10751 (1994); Stemmer, Nature 370: 389-391 (1994); Crameri et al., Nature 391: 288-291 (1998); Stemmer et al., U.S. Pat. No. 5,830,721, which are incorporated herein by reference. Gene shuffling or DNA shuffling is a method using in vitro homologous recombination of pools of selected mutant genes. For example, a pool of point mutants of a particular gene can be used. The genes are randomly fragmented, for example, using DNase, and reassembled by PCR. If desired, DNA shuffling can be carried out using homologous genes from different organisms to generate diversity (Crameri et al., supra, 1998). The fragmentation and reassembly can be carried out, for example, in multiple rounds, if desired. The resulting reassembled genes are a population of variants that can be used in the invention. Simultaneous incorporation of all of the encoding nucleic acids and all of the selected amino acid position changes can be accomplished by a variety of methods known to those skilled in the art, including for example, recombinant and chemical synthesis. Simultaneous incorporation can be accomplished by, for example, chemically synthesizing the nucleotide sequence for the region and incorporating at the positions selected for harboring variable amino acid residues a plurality of corresponding amino acid codons.

The biological activity of soluble CD26 is shared by any analogue or variant thereof that is capable of cleaving NH₂-terminal dipeptides from polypeptides with either proline or alanine at the penultimate position, or that possesses an immune epitope that is immunologically cross-reactive with an antibody raised against at least one epitope of the corresponding soluble CD26. The biological activity of placental soluble CD26 is shared by any analogue or variant thereof that is capable of cleaving NH₂-terminal dipeptides from polypeptides with either proline or alanine at the penultimate position, or that possesses an immune epitope that is immunologically cross-reactive with an antibody raised against at least one epitope of the corresponding placental soluble CD26. In one aspect of the invention, soluble CD26 and/or placental soluble CD26 is capable of cleaving VEGF at the CD26 recognition motif sequence. Yet in another aspect of the invention, soluble CD26 and/or placental soluble CD26 is capable of cleaving IL-2 at the CD26 recognition motif sequence.

As used herein, the terms “recognition motif” and “recognition motif sequence” are used interchangeably in the broadest sense and refer to NH₂-terminal dipeptides present in polypeptides with either proline or alanine at the penultimate position, which are cleavable with sCD26 and/or placental sCD26. Examples of biological polypeptides having such CD26 recognition motif sequence include VEGF, IL-2 and IL-6.

The term “VEGF” is used herein in the broadest sense and includes all naturally occurring VEGF molecules of any animal species, and all naturally occurring mutant and variant forms thereof, and its functional derivatives, such as amino acid sequence variants, which are capable of having the biological activity of VEGF and are capable of being cleaved by sCD26 and/or placental sCD26. The term “VEGF activity,” used herein, refers to the biological activity of naturally occurring VEGF that is capable of promoting selective growth of vascular endothelial cells.

The term “IL-2” is used herein in the broadest sense and includes all naturally occurring IL-2 molecules of any animal species, and all naturally occurring mutant and variant forms thereof, and its functional derivatives, such as amino acid sequence variants, which are capable of having the biological activity of IL-2 and are capable of being cleaved by sCD26 and/or placental sCD26.

The term “IL-2 activity,” used herein, refers to the biological activity of all naturally occurring IL-2, including but not limited to stimulating lymphocytes to activate and differentiate.

The term “native” or “native sequence” used herein in connection with CD26, soluble CD26 or any other polypeptide refers to a polypeptide that has the same amino acid sequence as a corresponding polypeptide derived from nature, regardless of its mode of preparation. Such native sequence polypeptide can be isolated from nature or can be produced by recombinant and/or synthetic means or any combinations thereof. The term “native sequence” specifically encompasses naturally occurring truncated or secreted forms (e.g., an extracellular domain sequence), naturally occurring variant forms (e.g., alternatively spliced forms) and naturally-occurring allelic variants of the full length polypeptides.

“Functional derivatives” include amino acid sequence variants, and covalent derivatives of the native polypeptides as long as they retain a qualitative biological activity of the corresponding native polypeptide. Amino acid sequence variants generally differ from a native sequence in the substitution, deletion and/or insertion of one or more amino acids anywhere within a native amino acid sequence. Deletional variants include fragments of the native polypeptides, and variants having N- and/or C-terminal truncations. Ordinarily, amino acid sequence variants will possess at least about 70% homology, preferably at least about 80%, more preferably at least about 90% homology, most preferably at least 91%, 92%, 93%, 94%, 95%, 96%, 97%, 98%, or 99% with a native polypeptide.

The term “treatment” is an intervention performed with the intention of preventing the development or altering the pathology of a disorder. Accordingly, “treatment” refers to both therapeutic treatment and prophylactic or preventative measures, wherein the object is to prevent or slow down (lessen) the targeted pathologic condition or disorder. Those in need of treatment includes those already inflicted the disorder, as well as those prone to having the disorder, or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. In treatment of an immune related disease, a therapeutic agent may directly alter the magnitude of response of a component of the immune response, or render the disease more susceptible to treatment by other therapeutic agents, e.g., antibiotics, antifungals, anti-inflammatory agents, chemotherapeutics, etc.

“Angiogenesis” is the formation of new blood vessels by sprouting from pre-existing ones. The generation of new blood vessels involves a multistep process, which includes the migration of vascular endothelial cells into tissue, followed by the condensation of such endothelial cells into vessels. Angiogenesis may be induced by an angiogenic agent or be the result of a natural condition.

Angiogenesis is involved in a variety of diseases or disorders. Some examples of pathological conditions involving angiogenesis include but are not limited to, macular degeneration; corneal neovascularization; ocular neovascular glaucoma; diabetic retinopathy; corneal graft rejection; vitamin A deficiency; Sjorgen's disease; acne rosacea; mycobacterium infections; bacterial and fungal ulcers; Herpes simplex infections; systemic lupus; rheumatoid arthritis; osteoarthritis; psoriasis; chronic inflammatory diseases (e.g., ulcerative colitis, Crohn's disease); hereditary diseases such as Osler-Weber Rendu disease and haemorrhagic teleangiectasia.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of cancers include breast cancer, colon cancer, squamous cell cancer, lung cancer, small-cell lung cancer, non-small cell lung cancer, prostate cancer, hepatocellular cancer, gastrointestinal cancer, pancreatic cancer, glioblastoma, cervical cancer, ovarian cancer, endometrial carcinoma, liver cancer, bladder cancer, cancer of the urinary tract, salivary gland carcinoma, kidney cancer, vulval cancer, thyroid cancer, renal cancer, carcinoma, melanoma, hepatic carcinoma, brain cancer and various types of head and neck cancer.

The “pathology” of a disease, such as tumor, includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, antibody production, auto-antibody production, complement production, infiltration of inflammatory cells (neutrophilic, eosinophilic, monocytic, lymphocytic) into cellular spaces, etc.

As used herein, the term “inflammatory disease” or “inflammatory disorder” are used interchangeably and refers to a disease or disorder in which a component of the immune system of a mammal causes, mediates or otherwise contributes to an inflammatory response contributing to morbidity in the mammal. Also included are diseases in which reduction of the inflammatory response has an ameliorative effect on progression of the disease and autoimmune diseases.

Examples of immune-related and inflammatory diseases, some of which are T cell mediated, include, without limitation, inflammatory bowel disease (such as Crohn's disease and ulcerative colitis), systemic lupus erythematosus, rheumatoid arthritis, juvenile chronic arthritis, gouty arthritis, rheumatoid spondylitis, spondyloarthropathies, systemic sclerosis (scleroderma), idiopathic inflammatory myopathies (dermatomyositis, polymyositis), Sjögren's syndrome, Wegener's granulomatosis (WG), systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, autoimmune diabetes, immune-mediated renal disease (glomerulonephritis, tubulointerstitial nephritis), kidney inflammation, demyelinating diseases of the central and peripheral nervous systems such as multiple sclerosis, idiopathic polyneuropathy, hepatobiliary diseases such as infectious hepatitis (hepatitis A, B, C, D, E and other nonhepatotropic viruses), autoimmune chronic active hepatitis, primary biliary cirrhosis, granulomatous hepatitis, and sclerosing cholangitis, inflammatory and fibrotic lung diseases (e.g., cystic fibrosis), gluten-sensitive enteropathy, Whipple's disease, autoimmune or immune-mediated skin diseases including bullous skin diseases, erythema multiforme and contact dermatitis, psoriasis, inflammatory skin diseases including atopic dermatitis, systemic scleroderma and sclerosis, asthma, allergic diseases of the lung such as eosinophilic pneumonia, idiopathic pulmonary fibrosis chronic pulmonary inflammatory disease, and hypersensitivity pneumonitis, transplantation associated diseases including graft rejection and graft-versus host disease, ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms, cerebral edema secondary to stroke, cranial trauma, hypovolemic shock, asphyxia, adult respiratory distress syndrome, acute-lung injury, Behcet's Disease, dermatomyositis, polymyositis, multiple sclerosis (MS), meningitis, encephalitis, uveitis, osteoarthritis, lupus nephritis, diseases involving leukocyte diapedesis, central nervous system (CNS) inflammatory disorder, Alzheimer's disease, multiple organ injury syndrome secondary to septicaemia or trauma, alcoholic hepatitis, bacterial pneumonia, antigen-antibody complex mediated diseases including glomerulonephritis, sepsis, sarcoidosis, immunopathologic responses to tissue/organ transplantation, inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis, etc.

The preferred indications include, without limitation, rheumatoid arthritis (RA), psoriasis, rheumatoid spondylitis, gouty arthritis; autoimmune diabetes, autoimmune hepatitis; multiple sclerosis (MS), asthma, systemic lupus erythematosus, Wegener's granulomatosis (WG), kidney inflammation, lupus nephritis, chronic pulmonary inflammatory disease, inflammatory bowel disease (IBD), Alzheimer's disease, diabetic retinopathy, age-related macular degeneration, corneal neovascularization and ocular allergy along with any disease or disorder that relates to inflammation and related disorders.

The term “subject” is a mammal. The term “mammal” as used herein refers to any animal classified as a mammal, including, without limitation, humans, domestic and farm animals, and zoo, sports or pet animals such horses, pigs, cattle, sheep, dogs, primates, cats, rodents, and ferrets, etc. In a preferred embodiment of the invention, the mammal is a human.

The term “antibody” is used in the broadest sense and specifically covers single monoclonal antibodies (including agonist, antagonist, and neutralizing antibodies) specifically binding a soluble CD26 of the present invention and antibody compositions with polyepitopic specificity.

The term “antagonist” is used in the broadest sense and includes any molecule that partially or fully blocks, inhibits, or neutralizes a biological activity of a soluble CD26 and/or placental soluble CD26 disclosed herein. In a similar manner, the term “agonist” is used in the broadest sense and includes any molecule that mimics a biological activity of a soluble CD26 and/or placental soluble CD26 disclosed herein. Suitable agonist or antagonist molecules specifically include agonist or antagonist antibodies or antibody fragments, fragments or amino acid sequence variants of soluble CD26 and/or placental soluble CD26.

The terms “specific binding”, “specifically binds” and “specific for” are used interchangeably and indicate that the variable regions of the antibodies recognize and bind sCD26 polypeptides exclusively (i.e., able to distinguish the polypeptide from other similar polypeptides despite sequence identity, homology or similarity found in the family of polypeptides), but may also interact with other proteins (for example, S. aureus protein A or other antibodies in ELISA techniques) through interactions with sequences outside of the variable region of the antibodies, and in particular, in the constant region of the molecule. Screening assays to determine binding specificity of an antibody are well known and routinely practiced in the art. Antibodies that recognize and bind fragments of the polypeptides of the invention are also contemplated, provided that the antibodies are specific for full-length sCD26 polypeptides. In one embodiment, the antibody specifically binds to soluble CD26 without significantly cross reacting with another antigen. In another embodiment the antibody is capable of specifically binding to placental sCD26, more preferably the antibody does not specifically bind to a soluble CD26 lacking the first 38 N-terminal amino acid residues. In yet another embodiment, the antibody specifically binds to the dimer of soluble CD26 and/or placental soluble CD26 but does not specifically bind to the monomer of soluble CD26 and/or placental soluble CD26. In another embodiment, the antibody recognizes a region of 4 amino acids of placental sCD26, more preferably a region of 6 amino acids of placental sCD26, and most preferably a region of 9 amino acids of placental sCD26.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include hybrid and recombinant antibodies produced by splicing a variable (including hypervariable) domain of an anti-sCD26 antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256: 495 (1975), or may be made by recombinant DNA methods such as described in U.S. Pat. No. 4,816,567. The “monoclonal antibodies” may also be isolated from phage libraries generated using the techniques described in McCafferty et al., Nature, 348: 552-554 (1990), for example.

Generally lymphocytes which produce the desired antibody are fused with an immortalized cell line using a suitable fusing agent. Immortalized cell lines or hybridomas are usually transformed mammalian cells, particularly myeloma cells of rodent, bovine and human origin. The hybridoma cells may be cultured in a suitable culture medium that preferably contains one or more substances that inhibit the growth or survival of the unfused immortalized cells. Preferred immortalized cell lines are those that fuse efficiently, support stable high level expression of antibody by the selected antibody producing cells and are sensitive to a medium such as HAT (hypoxanthine, aminopterin and thymidine). Examples of immortalized cell lines are murine myeloma lines which can be obtained from the American Type Culture Collection. Human myeloma and mouse-human heteromyeloma cell lines have also been described for the production of human monoclonal antibodies. (Kozbor, J. Immunol. 133: 3001 (1984))

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

“Humanized” forms of non-human (e.g., murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

“Antibody fragments” is used in the broadest sense and comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8 (10): 1057-1062, 1995); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and antigen-binding site. This region consists of a dimer of one heavy-chain and one light-chain variable domain in tight, non-covalent association. It is in this configuration that the three CDRs of each variable domain interact to define an antigen-binding site on the surface of the V_{H}—V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}—V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448, 1993.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable.

The term “prognosis” is used herein to refer to the prediction of the likelihood of cancer-attributable death or progression, including recurrence, metastatic spread, and drug resistance, of a neoplastic disease, such as breast cancer, or head and neck cancer. The term “prediction” is used herein to refer to the likelihood that a patient will respond either favorably or unfavorably to a drug or set of drugs, and also the extent of those responses, or that a patient will survive, following surgical removal of the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “effective amount” or “therapeutically effective amount” refers to an amount of a drug effective to treat and/or prevent a disease, disorder or unwanted physiological condition in a mammal. In the present invention, an “effective amount” of sCD26 and/or placental sCD26 may prevent, reduce, slow down or delay the onset of a disease or disorder such as cancer and inflammatory diseases, prevent or inhibit (i.e., slow to some extent and preferably stop) the development of a disease or a disorder such as cancer and inflammatory diseases; and/or relieve, to some extent, one or more of the symptoms associated with a disease or a disorder such as cancer and inflammatory diseases.

### B. DETAILED DESCRIPTION

The monoclonal antibody used in the method of the present invention may be prepared using procedures known in the art.

Decidual leukocytes were isolated from samples of decidual tissues according to the methods described in King et al. (Hum. Immunol., 24 (3); 195-205(1989) and the levels of VEGF produced by decidual leukocytes were measured using ELISA. The decidual leukocytes were then co-cultured with fetal trophoblast cells. The levels of VEGF were measured in the co-culture system using ELISA. Since the supernatant from cultured fetal trophoblast cells contains the placental sCD26, it was collected and concentrated before being used as an immunogen in mice for generating monoclonal antibody. The monoclonal antibody specific for the placental sCD26 was then prepared according to the procedures described in the manufacturer's instruction manual (StemCell Technologies, Canada, CLONACELL™-HY). The monoclonal antibodies from the hybridoma cells were screened for their ability to inhibit the function of placental sCD26. One of these antibodies was then immobilized onto a commercial agarose bead column and used to extract the placental sCD26 from the supernatant of cultured fetal trophoblast cells. The purified sCD26 was then separated on an SDS-polyacrylamide gel using gel electrophoresis and visualized by silver staining method and confirmed as placental sCD26, which lacks the first 28 N-terminal residues of the membrane CD26, by amino acid sequencing.

The isolated human placental sCD26, which lacks the first 28 N-terminal residues of the human membrane CD26, showed inhibition of VEGF activity and IL-2 activity. Furthermore, anti-placental sCD26 antibody was shown to block the inhibition of VEGF activity and IL-2 activity by placental sCD26.

Autoimmune diseases, such as rheumatoid arthritis, are associated with the presence of inflammatory agents such as IL-2. Some of the pro-inflammatory cytokines in rheumatoid arthritis also cause the production of VEGF, a growth factor that exacerbates the condition by stimulating the growth of new blood vessels, which supply nutrients and oxygen to the inflammatory cell mass found in arthritic joints. It has been reported that 75% of women with rheumatoid arthritis who became pregnant had an improvement in their symptoms, which generally lasted until the 4th month after delivery (Spector TD and Da Silva JA. (1992) Am J Reprod Immunol 28 (3-4): 222-225; Olsen NJ and Kovacs WJ. (2002) J Gend Specif Med 5 (4): 28; Ostensen M and Villiger PM. (2002) Transpl Immunol 9 (2-4): 155-160 and Straub RH., Buttgereit F., and Cutolo M. (2005) Ann Rheum Dis 64 (6): 801). It has now been shown that the presence of placental sCD26 in the periphery of pregnant women is capable of inhibiting VEGF. Thus CD26 is a therapeutic agent for chronic inflammatory conditions such as rheumatoid arthritis.

The invention provides methods of treatment, inhibition and prophylaxis by administration to a subject of an effective amount of a compound or pharmaceutical composition of the invention. In a preferred aspect, the compound is substantially purified (e.g., substantially free from substances that limit its effect or produce undesired side-effects).

Various delivery systems are known and can be used to administer a sCD26 of the invention, e.g., encapsulation in liposomes, microparticles, microcapsules, recombinant cells capable of expressing the compound, receptor-mediated endocytosis (see, e.g., Wu and Wu, J. Biol. Chem. 262: 44294432 (1987)). Methods of introduction include but are not limited to intradermal, intramuscular, intraperitoneal, intravenous, subcutaneous, intranasal, epidural, and oral routes. The sCD26 or compositions thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, such as sCD26, including its antibody, of the invention, care must be taken to use materials to which the protein does not absorb.

In another embodiment, the compound or composition can be delivered in a vesicle, in particular a liposome (see Langer, Science 249: 1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound or composition can be delivered in a controlled release system. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249: 1527-1533 (1990)).

The formulations to be used for in vivo administration must be sterile. This is readily accomplished by filtration through sterile filtration membranes, prior to or following lyophilization and reconstitution.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG. Suitable pharmaceutical excipients also include starch, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Therapeutic compositions herein generally are placed into a container having a sterile access port, for example, an intravenous solution bag or vial having a stopper pierceable by a hypodermic injection needle.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a sCD26 or agonist or antagonist thereof is employed, normal dosage amounts may vary from about 10 ng/kg to up to 100 mg/kg of mammal body weight or more per day, preferably about 1 mg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a sCD26 is desired in a formulation with release characteristics suitable for the treatment of any disease or disorder requiring administration of the sCD26, microencapsulation of the sCD26 is contemplated. Microencapsulation of recombinant proteins for sustained release has been successfully performed with human growth hormone (rhGH), interferon-(rhIFN-), interleukin-2, and MN rgp120. Johnson et al., Nat. Med., 2: 795-799 (1996); Yasuda, Biomed. Ther., 27: 1221-1223 (1993); Hora et al., Bio/Technologs 8: 755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 141.

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention.

Antibodies of the invention can be used to assay protein levels in a biological sample using classical immunohistological methods known to those of skill in the art (e.g., see Jalkanen, et al., J. Cell. Biol. 101: 976-985 (1985); Jalkanen, et al., J. Cell. Biol. 105: 3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin.

### EXAMPLES

1. Isolation of Decidual Leukocytes and Fetal Trophoblast Cells

Decidual Leukocytes

Samples were obtained from elective termination of first trimester pregnancies from Addenbrookes Hospital, Cambridge, Mass. Preparation of maternal decidual leukocytes was carried out as described (King et al., Hum. Immunol., 24 (3); 195-205(1989)) with minor modifications.

Samples of decidual tissues were sorted by macroscopical examination and washed in cold RPMI 1640 (Invitrogen, USA, Catalog No. 21875-034) for 10-20 minutes before being minced into small pieces using surgical blades. Approximately 10 grams of minced tissue was digested in 25 ml of RPMI 1640 containing 10% fetal calf serum (FCS) (Harlan Sera-Lab, UK, Catalog No. S-0001A), 2 ml of Collagenase (10 mg/ml, Sigma, U.S., Catalog No. C5138), and 0.5 ml DNAse I (3 mg/ml, Sigma D5025) at 37° C. on a roller incubator for 30 minutes. The sample tube was centrifuged briefly to pellet pieces of tissue and the cell-containing supernatant was filtered through a 100 μm filter (Becton Dickinson, USA, Catalog No. 352360). The flow-through was centrifuged at 650×g for 5 minutes to pellet cells. The supernatant was added back to the tissue which had been passed through a 10 ml pipette a few times to further break up the tissue. The mixture was incubated for another 10 minutes at 37° C. The sample tube was then centrifuged briefly to pellet tissues and supernatant was filtered through 100 μm filter. Filtered supernatant was centrifuged at 650×g for 5 minutes to pellet the cells. The cell pellet was resuspended in 15 ml of PBS (Current Protocols in Molecular Biology, Wiley Press, page 4.2.3, 1996) containing 2% FCS and 0.1% azide before being overlaid onto 15 ml of LYMPHOPREP™, a ready-made, sterile and endotoxin tested solution suitable for the purification of human mononuclear cells (Axis-Shield Diagnostics, Norway, Catalog No. 1114545). The tube was centrifuged at 710×g for 20 minutes without brake and the cells at the interface were collected and washed once in RPMI 1640 (10% FCS). Cells prepared in this way usually consisted of approximately 60% NK cells (CD56⁺ CD 16⁻), 15-20% macrophages (CD14⁺), 10% T cells and other stromal cells.

Fetal Trophoblast Cells

Fragments of placental tissue were identified macroscopically and washed in RPMI 1640 medium for a few minutes. The tissue was scraped using scalpel blades and then digested in 20 ml of prewarmed (37° C.) 0.25% trypsin (Becton Dickinson, USA, Catalog No. 215240), 0.02% EDTA for 8-9 minutes on a hotplate with stirring. 20 ml of HAMS F12 (LIFE Technologies, USA, Catalog No. 074-90587) (20% NCS (Invitrogen, USA, Catalog No. 16010-167)) was added to the solution to stop the trypsinization. The solution was then filtered through gauze and centrifuged in 50 ml tubes at 450×g to pellet the cells. The cell pellet was resuspended in 10 ml of HAMS F12 and the cell solution was overlaid onto 10 ml of LYMPHOPREP™, a ready-made, sterile and endotoxin tested solution suitable for the purification of human mononuclear cells (Axis-Shield Diagnostics, Norway, Catalog No. 1114545), and centrifuged at 710×g for 20 minutes. Cells at the interface were recovered and washed once with 10 ml of HAMS medium. To deplete placental macrophages, the cell pellet was resuspended in 3 ml of HAMS and seeded onto a petri dish and incubated for 20 minutes at 37° C. Cells in the supernatant were recovered by being centrifuged at 600×g for 5 minutes.

2. Concentration of Supernatant from Cultured Fetal Trophoblast Cells

Fetal trophoblast cells were isolated as described above. Cells were cultured in RPM-I1640 medium plus 10% fetal calf serum (FCS) at 1×10⁶ cell/ml at 37° C. overnight. The supernatant was collected and centrifuged at 1,000×g for 5 minutes to pellet the cell debris before loaded onto a centrifugal filter device CENTRICON® (Millipore, YM-10) and centrifuged at 2,000×g for around 1 hour. The volume was usually reduced to 1/10 of the original volume.

3. Co-Culture of Decidual Leukocytes with Placental Trophoblasts

This assay was used to determine the presence of sCD26 in the cultured trophoblasts supernatant.

Decidual leukocytes and placental trophoblasts were isolated as described above. A trophoblasts cell line, JEG, was cultured in RPMI-1640 medium (10% FCS) as negative control. 100 μl of leukocytes (3×10⁶ cells/ml) were seeded in each well of a 96-well U-bottom plate with or without 100 μl of the isolated trophoblasts or negative control cells JEG (1×10⁶ cells/ml). After overnight incubation, the culture supernatants were harvested and stored at “70° C. for later ELISA assay. 100 μl of each stored supernatant was thawed and used in a VEGF ELISA assay (R&D, USA, Catalog No. DY293) according to the manufacturer's instruction manual.

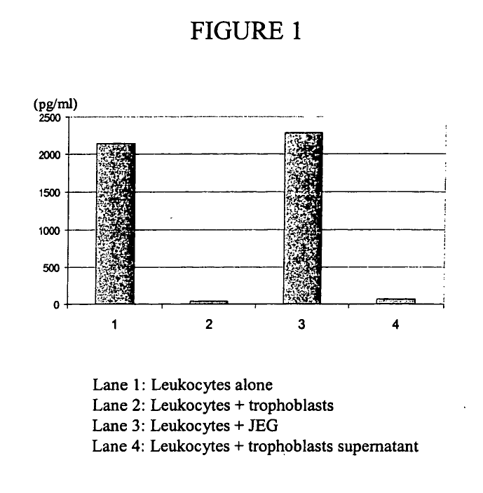

FIG. 1 shows the results of the assay. Lane 1 shows the VEGF produced by decidual leukocytes that was detected in the culture supernatant by the VEGF ELISA assay. Lane 2 shows the disappearance of the VEGF in the culture supernatant when the decidual leukocytes are co-cultured with the isolated trophoblasts in the same container. As a negative control, lane 3 shows the production of VEGF when the decidual leukocytes are co-cultured with the cell line JEG.

Accordingly, the inhibition of VEGF detection is specifically due to the co-culture with the isolated trophoblasts. Lane 4 shows that the effect of trophoblasts on the detection of VEGF can also be repeated by the addition of only the trophoblasts culture supernatant.

The assay result showed that sCD26, which abolishes the detection of VEGF in an ELISA assay, was present in the cultured trophoblasts supernatant.

4. Screening of Anti-Placental sCD26 Monoclonal Antibody Produced by Mouse Hybridoma

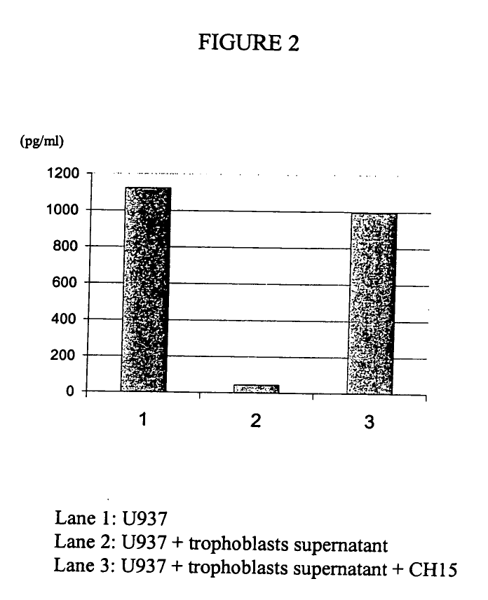

The spleen cells from the immunized mouse were fused with SP2/0 cells according to the manufacturer's procedure for generation of hybridoma (StemCell Technologies, CLONACELL™-HY, Catalog No. 03800). After the hybridoma cells grew up, 100 μl of supernatant from each hybridoma was used in the 96-well plate containing 50 μl of the commonly used cell line U937 at 0.5×10⁶ cells/ml (ATCC, CRL1593) plus 50 μl of fetal trophoblast cell supernatant. U937 cell line was used since decidual leukocytes are rather difficult to obtain. U937 also produces VEGF (see FIG. 2, lane 1) and similar to the results shown above in FIG. 1, the detection of VEGF is abolished when U937 was co-cultured with trophoblasts supernatant (see FIG. 2, lane 2). Only the hybridoma which secrets the monoclonal antibody specific for the VEGF inhibitor will reverse the inhibition of VEGF detection in this assay. Accordingly, few of these hybridoma cells were successfully selected. One of these antibodies, designated as CH15, was selected for further experiments. FIG. 2, lane 3 shows that when CH15 was added to the co-culture of U937 with trophoblasts supernatant, it restored the VEGF production by U937. This indicates that CH15 binds to the sCD26 to block its activity as shown by the detection of VEGF.

5. Purification of CH15, Anti-Placental sCD26 Monoclonal Antibody, and Immobilization onto Agarose Gel

Monoclonal antibody with specificity for placental sCD26 in the hybridoma cells culture supernatant was purified by protein-A column. Half gram of protein-A sepharose (Sigma, USA, Catalog No. P3391) powder was hydrated in 1 ml of PBS buffer (pH 7.4) and loaded into a plastic column (Bio-Rad, USA, Catalog No. 732-1010). The column was washed with 10 ml of PBS buffer (pH 8). The hybridoma supernatant was then passed through the protein-A column slowly allowing antibody to bind to the protein-A. Afterwards, the protein-A column was washed a few times with buffers and antibody was eluted from the column with 100 mM glycine (pH 3) (Antibodies, Harlow, E. and Lane, D., Cold Springs Harbor Lab Press, 1988, p. 310).

6. Immunoprecipitation of Placental sCD26 from Fetal Trophoblasts Supernatant

200 μg of CH15, a placental sCD26-specific antibody, was immobilized onto agarose gel for sCD26 immunoprecipitation according the manufacturer's instruction (SEIZE™ primary immunoprecipitation kit, Pierce Catalog No. 45335). Concentrated supernatant from cultured fetal trophoblast cells was then incubated with the antibody-conjugated agarose gel for overnight at 4° C. to allow antibody to bind placental sCD26. The agarose gel was then washed few times with buffer and proteins were then eluted from antibody-conjugated agarose gel with elution buffer.

7. Visualization of Immunoprecipitated Placental sCD26 on a Polyacrylamide Gel

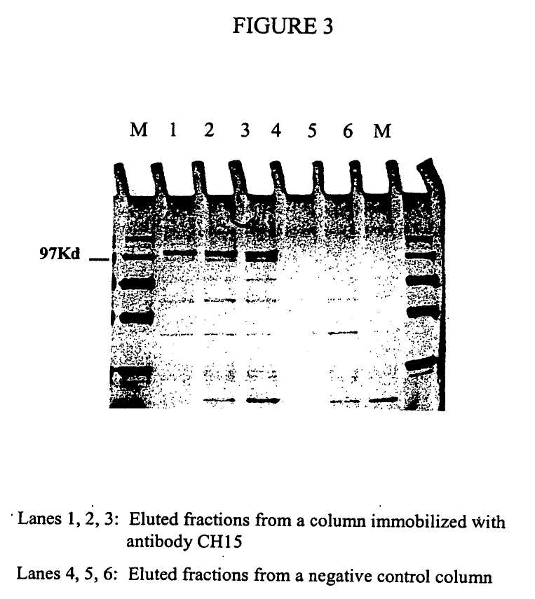

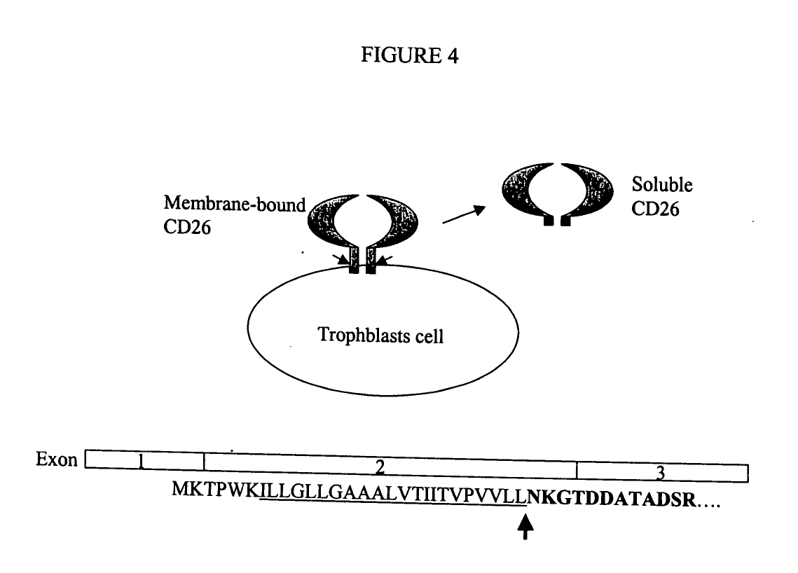

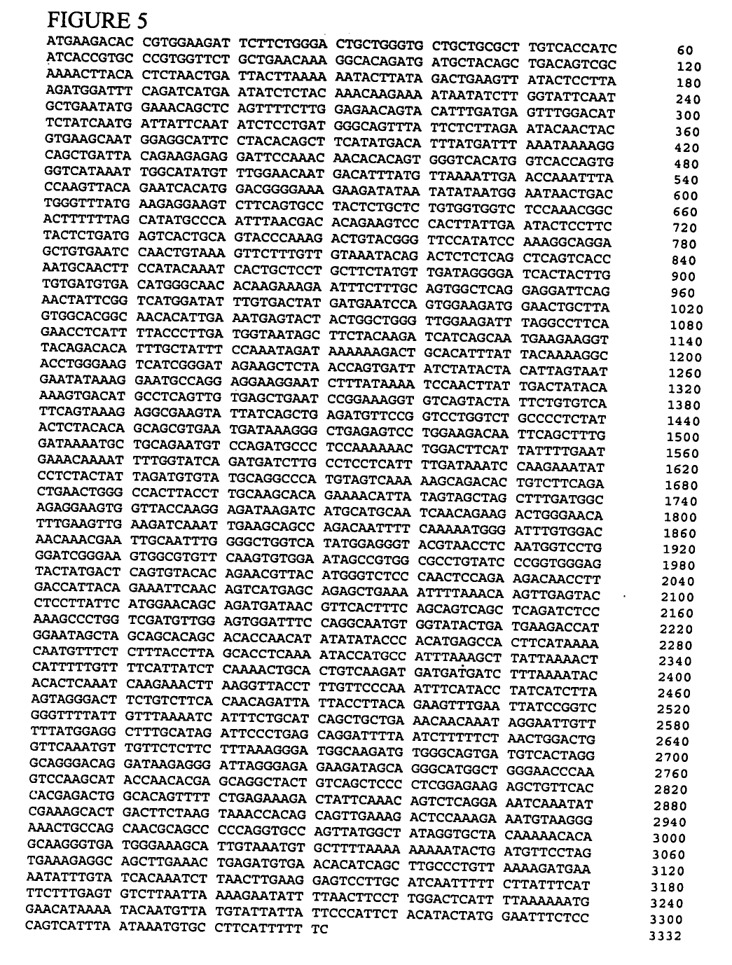

20 μl of eluted sample was mixed with 5 μl of gel loading dye and boiled for 5 minutes. The sample was loaded into a 10% SDS polyacrylamide (SDS-PAGE) gel and electrophoresis was run at 100 volts for 1.5 hours in Tris-Glycine buffer (Current Protocols in Molecular Biology, Wiley Press, page A.2.5, 1996) with 2 mM mercaptoacetic acid (Sigma, USA, Catalog No. T-6750). After the gel electrophoresis, the proteins in the gel were blotted to a PVDF membrane (Bio-Rad, USA, Catalog No. 162-0185) in CAPS buffer (19 mM CAPS (Sigma, USA, Catalog No. C-4142), 5 mM DTT (Sigma, USA, Catalog No. D9163), 10% Methanol, pH 11) at 50 volts for one hour. After blotting, the membrane was stained with Coomassie Blue R-250 (Bio-Rad, USA, Catalog No. 161-0435) or stained by silver staining (Bio-Rad, USA, Catalog No. 161-0449) according to the manufacturer's instructions. To visualize the immunoprecipitated proteins by CH15. FIG. 3 shows the protein bands visualized by silver staining. Lanes 1, 2 and 3 are eluted fractions from the antibody-conjugated agarose gel. Lanes 4, 5 and 6 are eluted fractions from negative control. After staining, a protein band of approximately 110 kDa, as shown in lanes 1-3, was cut out and sequenced.

The sequencing data identified the immunoprecipitated protein to be a soluble form of placental CD26. Accordingly, the antibody CH15 specifically binds to the placental sCD26 present in the supernatant of cultured trophoblast cells.

8. Effect of Placental sCD26 on the Proliferation of Human Umbilical Venous Endothelial Cell (HUVEC)

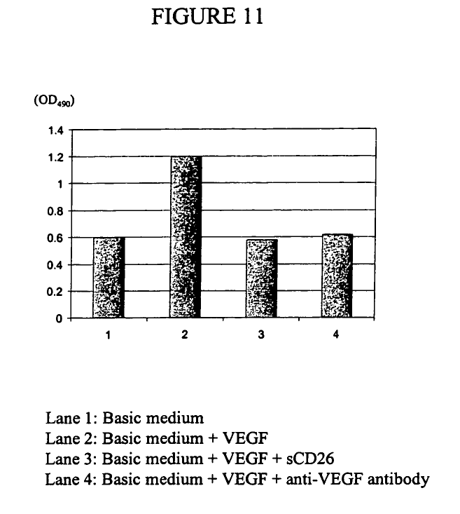

This assay is designed to determine whether placental sCD26 inhibits the proliferation of human umbilical vein endothelial cells. The proliferation of HUVECs is a well documented response to the stimulation by VEGF. Without the supply of VEGF, HUVECs in culture stop growing and gradually die off. In this experiment, HUVECs were cultured in basic medium with or without VEGF to show the dependence of HUVEC proliferation on VEGF (FIG. 11, lanes 1 and 2). Since HUVEC proliferation by VEGF stimulation is essential in angiogenesis, a test compound that results in a the inhibition of VEGF activity in the present assay can be said to have anti-angiogenesis properties.

HUVECs were isolated as described with minor modification (Jaffe, E. A. et al., J. Clin. Invest. 52 (11): 2745-2756, 1973). Human umbilical cord was collected into 150 ml of PBS buffer containing fungizone at 1 μg/ml (Gibco, USA, Catalog No. 15295-017). The cord was washed with sterile PBS and the damaged ends were cut off with surgical blade. The vein was located and cannulated with a sterile Kwill filling tube (Avon Medicals, UK, Catalog No. E910) at both ends of the cord. The umbilical cord was tied up at the cannulated region with sterile thread. The cord blood was then flushed out with 100 ml PBS and the 20 ml of PBS was gently flushed back and forth between two 20 ml syringes (Becton Dickinson, USA, Catalog No. 300613). After flushing the cord thoroughly, the excess PBS was removed. 10 ml of Collagenase solution was added at 10 μg/ml (Sigma, USA, Catalog No. C-9891) and the ends of cannulas were plugged. The cord was then placed in prewarmed beaker containing PBS for 10 minutes. The Collagenase solution in the cord was gently flush back and forth between two syringes and the flow-through was collected in a 50 ml centrifuge tube. The cord was then washed with 10 ml of Medium-199 medium (Sigma, USA, Catalog No. M-7528) into the same tube. The tube was centrifuged at 200×g for 5 minutes to collect the HUVEC. The cells were resuspended in 10 ml of Endothelial cell growth medium (PromoCell, USA, Catalog No. C22010) and cultured in incubator at 37° C.

To show the inhibition of VEGF activity by placental sCD26, VEGF was incubated with placental sCD26 before adding to the culture of HUVECs. Cells were resuspended in Medium-199 (Sigma, USA, Catalog No. M-7528) at 2×10⁵ cells/ml and 50 μl of it was used per well in a 96-well flat bottom plate (Becton Dickinson, USA, Catalog No. 353072). VEGF was diluted in Medium-199 medium to give 1 μg/ml. Digestion of VEGF by sCD26 was illustrated by incubating VEGF (1 μg/ml) with equal volume of 10× concentrated trophoblasts supernatant at 37° C. for one hour. After digestion, the VEGF solution was diluted to 20 μg/ml with assay medium (Medium-199 plus 10% FCS and 10 mM HEPES) and 50 μl of it was placed per well. As a control, VEGF (1 μg/ml) was incubated with anti-VEGF antibody (R&D MAB293, 10 μg/ml) at room temperature for one hour (FIG. 11, lane 4). After mixture of HUVEC and VEGF, the plate was incubated at 37° C. for three days before conducting the cell proliferation assay (Promega, USA, Catalog No. G3580).

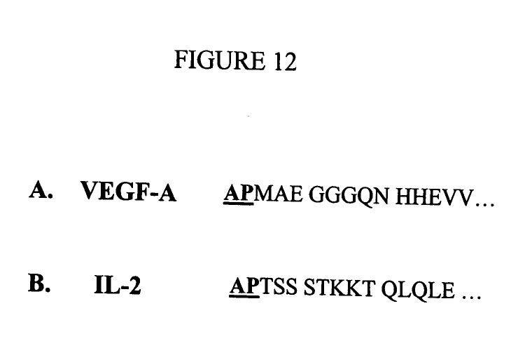

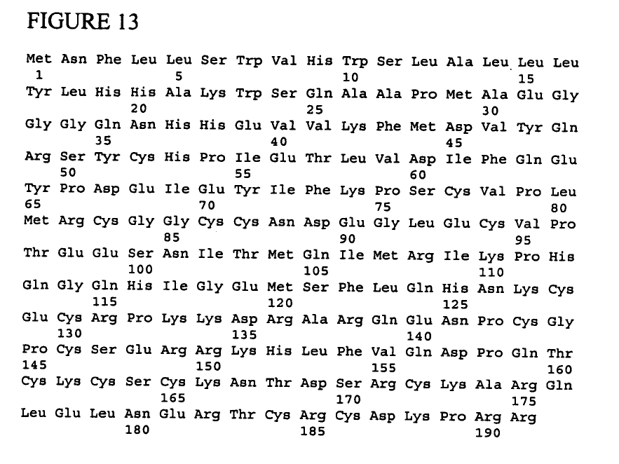

FIG. 12(A) shows the first 15 amino acid sequence from the N-terminal of mature and secreted VEGF-A. FIG. 13 shows the amino acid sequence of the VEGF-A (SEQ ID NO: 7) as found in the Genebank (Accession number M32977). The recognition motif sequence alanine-proline (“AP”) for placental sCD26 in VEGF-A is underlined. The VEGF-A is cleaved by placental sCD26 after the proline residue.

The result of this assay showed that once the VEGF is digested by placental sCD26, it loses its activity in stimulating KUVEC proliferation (FIG. 11, lane 3). Accordingly, placental sCD26 has anti-angiogenesis property.

9. Inhibition of IL-2 Activity by Placental sCD26

CTLL-2 cells (ATCC, TIB214) constitutively express IL-2 receptors and depend entirely on the presence of IL-2 for their growth. These cells are used in the present assay to show that placental sCD26 is capable of inhibiting the IL-2. The activity of IL-2 is determined by measuring the cell proliferation of CTLL-2 cells.

For this assay, CTLL-2 (0.1×10⁶ cells/ml) was washed twice with RPMI-1640 and used 50 μl per well. Recombinant IL-2 (PromoCell, Germany, Catalog No. C-61201) was used at final concentration 10 pg/ml. Treatment of IL-2 with placental sCD26 was done by mixing IL-2 (1 ng/ml) with equal volume of 10× concentrated trophoblasts supernatant for 1 hour at 37° C. After treatment, the concentration of IL-2 was adjusted to 20 ng/ml and use 50 μl per well. Plate was incubated at 37° C. for 3 days before measuring cell proliferation at OD490 (Promega, USA, Catalog No. G3580).

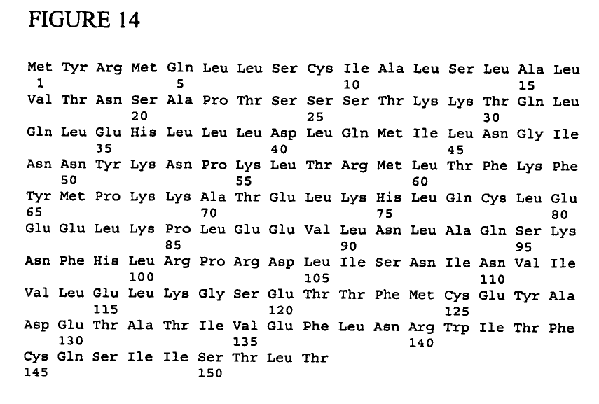

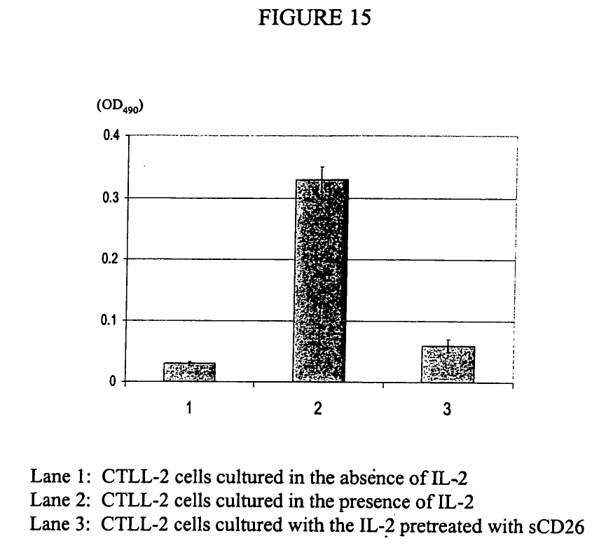

FIG. 15 shows the results of this experiment. The cell proliferation of CTLL-2 cells cultured in the absence and presence of IL-2 are shown in lanes 1 and 2, respectively. Without the supply of IL-2, CTLL-2 cells die off quickly as shown in FIG. 15, lane 1. IL-2 contains the alanine-proline (“AP”) motif recognized by placental sCD26 and is cleaved by placental sCD26 after the proline residue. The recognition motif sequence for placental sCD26 in IL-2 is underlined in FIG. 12(B). FIG. 14 shows the amino acid sequence of the IL-2 (SEQ ID NO: 8) as found in the Genebank (Accession number V00564).

Lane 3 of FIG. 15 shows that the treatment of IL-2 with placental sCD26 by incubating IL-2 in the trophoblasts supernatant rendered IL-2 inactive as indicated by the loss of its activity to stimulate CTLL-2 proliferation.

It is well known in the art that IL-2 is a pro-inflammation cytokine in human immune system. Accordingly, a placental soluble CD26 has an anti-inflammatory property since it inhibits the IL-2 activity.

10. Inhibition of VEGF by Soluble CD26 Present in Pregnant Women's Blood Serum

Levels of sCD26 in serum from male, non-pregnant female and pregnant subjects were compared by measuring how much each serum could mediate cleavage of exogenous VEGF.

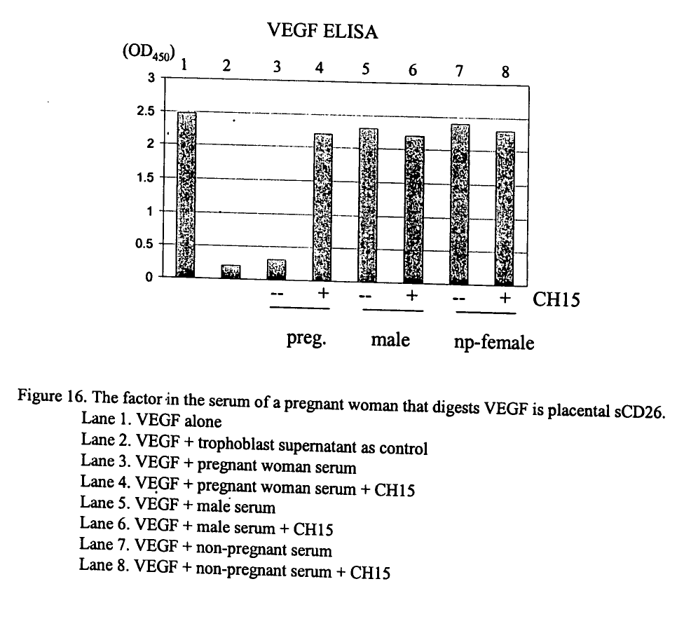

Five millilitre of human blood was collected and centrifuged at 700×g for 10 minutes. The top layer serum was aliquoted and stored at −20° C. for later use. Sera were thawed and incubated with anti-placental sCD26 antibody CH15 at 10 μg/ml for 30 minutes before mixed with VEGF at 0.1 ng/ml at 37° C. overnight. VEGF ELISA was performed on these reaction mixtures according the manufacturer's instruction manual R & D Systems, Inc. The results are shown in FIG. 16.

Pregnant woman's serum contains a factor that prevents the detection of VEGF in ELISA. This factor can be neutralized by monoclonal antibody CH15 (comparing Lanes 3 and 4). Since CH15 is a specific antibody for placental sCD26, it is circulating sCD26 from the placenta that prevents the detection of VEGF. This factor doesn't exist in the serum of either male or non-pregnant female blood (Lane 5 to 8).

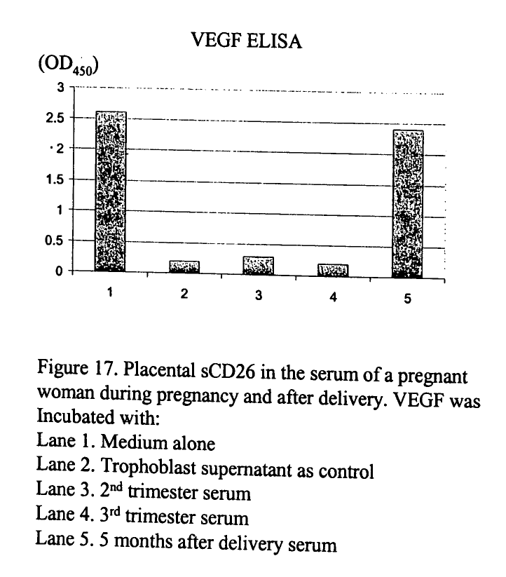

Five millilitre of blood was collected from a pregnant woman at the 2^{nd}, 3^{rd} trimester and 5 months after her delivery. Sera were made and VEGF ELISA was performed as described above. The results are shown in FIG. 17. Sera from the 2^{nd} and 3^{rd} trimester of the pregnancy contains placental sCD26 that prevents the detection of VEGF in ELISA (Lane 3 and 4). But 5 months after delivery, serum from the same woman no longer contains detectable activity of placental sCD26.

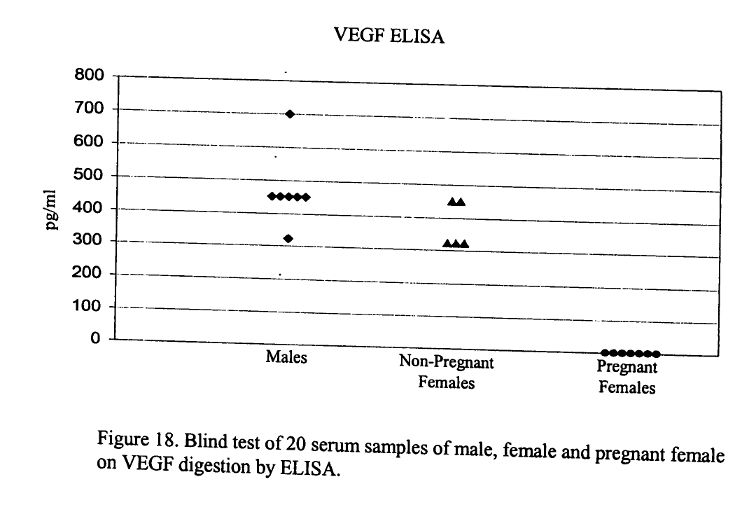

In a blind test five millilitre of blood was collected from 20 individuals of either males or females or pregnant females. Each sample was marked only by a number. 300 pg/ml VEGF was incubated overnight at 37° C. with venous sera from male (n=7), non-pregnant female (n=5) and pregnant female (n=7) volunteers and then VEGF levels were measured by ELISA by the method set forth above. After the experiment, the results were matched to the records of individual's gender and pregnancy status. The results are shown in FIG. 18. There were 7 samples collected from pregnant women and VEGF disappeared after incubated with these sera. All the other sera, either from male or non-pregnant female, did not have this effect on VEGF. The difference between non-pregnant and pregnant females was compared using the non-paired Students t-test and was found to be significant (p<0.0001).

What is claimed is:

1. A method of inhibiting VEGF activity, comprising contacting VEGF with an effective amount of soluble CD26 or a biologically active variant of soluble CD26.

2. A method of inhibiting angiogenesis in a mammalian subject, comprising administering to said subject a therapeutically effective amount of soluble CD26 or a biologically active variant of soluble CD26.

3. A method of treating a disease or a condition associated with angiogenesis in a mammalian subject, comprising administering to said subject a therapeutically effective amount of soluble CD26 or a biologically active variant of soluble CD26.

4. The method of claim 3 wherein said disease is cancer.

5. The method of claim 4 wherein said cancer is selected from the group consisting of lymphoma, leukemia, breast cancer, colon cancer, squamous cell cancer, lung cancer, small-cell lung cancer, non-small cell lung cancer, prostate cancer, hepatocellular cancer, gastrointestinal cancer, pancreatic cancer, glioblastoma, cervical cancer, ovarian cancer, endometrial carcinoma, liver cancer, bladder cancer, cancer of the urinary tract, salivary gland carcinoma, kidney cancer, vulval cancer, thyroid cancer, renal cancer, carcinoma, melanoma, hepatic carcinoma and brain cancer.

6. The method of claim 3 wherein said condition is selected from the group consisting of rheumatoid arthritis, macular degeneration, psoriasis, diabetic retinopathy, ocular neovascular glaucoma, corneal graft rejection, vitamin A deficiency, Sjorgen's disease, acne rosacea, mycobacterium infections, bacterial and fungal ulcers, Herpes simplex infections, systemic lupus, osteoarthritis, ulcerative colitis, Crohn's disease, Osler-Weber Rendu and haemorrhagic teleangiectasia.

7. The method of claim 2 wherein said mammalian subject is human.

8. The method according to claim 2, in which the soluble CD26 is administered permucosally, orally, enterally, percutaneously, subcutaneously, transdermally, intravenously, by aspiration, by suppository, by instillation, endoscopically, intratracheally, intralesionally, intratumorally, intramuscularly or via mucous membranes such as in a nasal spray.

9. A method of inactivating VEGF comprising cleaving said VEGF after a recognition motif sequence.

10. The method of claim 9 wherein said recognition motif sequence comprises alanine-proline.

11. A pharmaceutical composition comprising soluble CD26 or a pharmaceutically acceptable salt thereof, together with at least one pharmaceutically acceptable carrier.

12. A method of inhibiting IL-2 activity, comprising contacting IL-2 with an effective amount of soluble CD26 or a biologically active variant of soluble CD26.

13. A method of inactivating IL-2 comprising cleaving said IL-2 after a recognition motif sequence.

14. The method of claim 13 wherein said recognition motif sequence comprises alanine-proline.

15. A method of inhibiting inflammation in a mammalian subject, comprising administering to said subject a therapeutically effective amount of soluble CD26 or a biologically active variant of soluble CD26.

16. A method for the treatment of an inflammatory, immune or autoimmune disease in a mammalian subject, comprising administering to said subject a therapeutically effective amount of soluble CD26 or a biologically active variant of soluble CD26.

17. The method of claim 15 wherein said mammalian subject is human.

18. The method according to claim 15, in which the soluble CD26 is administered permucosally, orally, enterally, percutaneously, subcutaneously, transdermally, intravenously, by aspiration, by suppository, by instillation, endoscopically, intratracheally, intralesionally, intratumorally, intramuscularly or via mucous membranes such as in a nasal spray.

19. The method of claim 16 wherein said inflammatory, immune or autoimmune disease is selected from the group consisting of inflammatory bowel disease (such as Crohn's disease and ulcerative colitis), systemic lupus erythematosus, rheumatoid arthritis, juvenile chronic arthritis, gouty arthritis, rheumatoid spondylitis, spondyloarthropathies, systemic sclerosis (scleroderma), idiopathic inflammatory myopathies (dermatomyositis, polymyositis), Sjogren's syndrome, Wegener's granulomatosis (WG), systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, autoimmune diabetes, immune-mediated renal disease (glomerulonephritis, tubulointerstitial nephritis), kidney inflammation, demyelinating diseases of the central and peripheral nervous systems such as multiple sclerosis, idiopathic polyneuropathy, hepatobiliary diseases such as infectious hepatitis (hepatitis A, B, C, D, E and other nonhepatotropic viruses), autoimmune chronic active hepatitis, primary biliary cirrhosis, granulomatous hepatitis, and sclerosing cholangitis, inflammatory and fibrotic lung diseases (e.g., cystic fibrosis), gluten-sensitive enteropathy, Whipple's disease, autoimmune or immune-mediated skin diseases including bullous skin diseases, erythema multiforme and contact dermatitis, psoriasis, inflammatory skin diseases including atopic dermatitis, systemic scleroderma and sclerosis, asthma, allergic diseases of the lung such as eosinophilic pneumonia, idiopathic pulmonary fibrosis, chronic pulmonary inflammatory disease, and hypersensitivity pneumonitis, transplantation associated diseases including graft rejection and graft-versus host disease, ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms, cerebral edema secondary to stroke, cranial trauma, hypovolemic shock, asphyxia, adult respiratory distress syndrome, acute-lung injury, Behcet's Disease, dermatomyositis, polymyositis, multiple sclerosis (MS), meningitis, encephalitis, uveitis, osteoarthritis, lupus nephritis, diseases involving leukocyte diapedesis, central nervous system (CNS) inflammatory disorder, Alzheimer's disease, multiple organ injury syndrome secondary to septicaemia or trauma, alcoholic hepatitis, bacterial pneumonia, antigen-antibody complex mediated diseases including glomerulonephritis, sepsis, sarcoidosis, immunopathologic responses to tissue/organ transplantation, inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis.

20. The method of claim 16 wherein said inflammatory or autoimmune disease is selected from the group consisting of rheumatoid arthritis (RA), psoriasis, rheumatoid spondylitis, gouty arthritis, autoimmune diabetes, autoimmune hepatitis, multiple sclerosis (MS), asthma, systemic lupus erythematosus, Wegener's granulomatosis (WG), kidney inflammation, lupus nephritis, chronic pulmonary inflammatory disease, inflammatory bowel disease (IBD), Alzheimer's disease, diabetic retinopathy, age-related macular degeneration, corneal neovascularization and ocular allergy.

21. The method of claim 1 wherein said soluble CD26 is placental soluble CD26.

22. An antagonist antibody that specifically binds to and inhibits soluble CD26 or a biologically active variant of soluble CD26.

23. The antibody of claim 22 wherein said soluble CD26 is placental soluble CD26 and said biologically active variant of soluble CD26 is biologically active variant of placental soluble CD26.

24. The antibody of claim 22 wherein said soluble CD26, said placental soluble CD26, said biologically active variant of soluble CD26 or said biologically active variant of placental soluble CD26 is a dimer.

25. The antibody of claim 22 which is a monoclonal antibody.

26. The antibody of claim 22 which is a humanized antibody.

27. The antibody of claim 22 which is an antibody fragment.

28. The antibody of claim 22 which is labeled.

29. A hybridoma cell which produces the antibody of claim 22.


SEQUENCE LISTING (8 sequences)

SEQ ID NO: 1 - DNA, 3332 bp, homo sapiens
atgaagacaccgtggaagattcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttattgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcaccacgagactggcacagttttctgagaaagactattcaaacagtctcaggaaatcaaatatcgaaagcactgacttctaagtaaaccacagcagttgaaagactccaaagaaatgtaagggaaactgccagcaacgcagcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttc

SEQ ID NO: 2 - protein, 766 aa, Homo sapiens [sequence omitted]

SEQ ID NO: 3 - DNA, 2217 bp, Homo sapiens
aacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttattgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 4 - protein, 738 aa, Homo sapiens
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLIDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 5 - DNA, 2283 bp, Mus musculus
atgaagacaccgtggaaggttcttctgggactgcttggtgtcgctgcgcttgtcaccatcatcaccgtgccaatagttctgctgagcaaagatgaagcggcagctgacagccgcagaacgtattcactagctgactatttaaagagtacctttcgggtcaagtcctactctttgtggtgggtttcagactttgaatacctctacaaacaagagaacaatatcttgctgctcaatgctgaacatggaaacagctccattttcttggagaacagtacctttgaaagctttggatatcattcagtgtcacctgaccgactgtttgttctcttggaatacaactacgtgaagcaatggagacattcctacacagcttcatacaacatttatgatgtgaataaaagacagctgatcacagaagagaagattccaaataatacacagtggatcacatggtcaccagaaggtcataagttggcatatgtctggaagaatgatatttacgttaaagttgaaccacacttacctagtcataggatcacatcgacaggagaagaaaatgtaatatataatggaataactgactgggtttatgaagaggaagtcttcggtgcctactctgcactgtggtggtctccaaacaacacgtttctagcttatgcccagtttaacgacacaggagtgccgctcattgaatactccttctattctgatgagtcactgcagtaccccaagacagtgtggattccatacccaaaggcaggagctgtgaatccaactgtaaagttctttattgtaaatatagactctctcagctcatcctctagtgcggctcccatccaaatccctgctcctgcatctgtggcaagaggggatcactatttatgtgatgtggtgtgggctacagaagaaagaatttcactacagtggctcaggaggattcagaactattccgtgatggctatctgtgactatgataagatcaacctaacgtggaactgtccatccgagcagcagcatgttgaaatgagtaccacaggctgggtcggaagatttaggcccgcagaacctcacttcacctctgatggaagcagcttctataagatcatcagcgacaaagatggctacaaacacatctgccacttcccgaaagataagaaagactgtacatttattacaaaaggagcctgggaagtcattagtatcgaagctctgaccagcgattatctatactacattagtaaccaatataaagaaatgccaggaggaagaaatctctataaaattcaacttactgaccacacaaatgtgaagtgccttagttgtgacctgaatccagaaagatgtcagtattatgcggtatcatttagtaaagaggcaaagtactatcagctgggatgttggggccccggtctgcccctctacactctacatcgtagcacggatcataaagagctgcgagtcctggaagacaattctgctttggatagaatgctgcaggatgtccagatgccttcaaaaaaattggacttcattgttttgaatgaaacaagattttggtatcaaatgatcttgccccctcattttgataaatccaagaaatatcctctactattagatgtatatgcaggtccctgtagtcaaaaagcagatgcttccttcagactgaactgggccacttaccttgcaagtacagaaaacatcatagtagctagctttgacggcagaggaagtggttaccaaggagataagatcatgcatgcaatcaacagaagattgggaacactggaagttgaagatcaaattgaagcagccaggcaatttgtaaaaatgggatttgtggatagcaagcgagttgcaatttggggctggtcatatggagggtatgtaacctcaatggtcctgggatcgggaagtggcgtgttcaagtgcggaatagctgtggcacctgtgtcacggtgggagtactatgactcagtgtacacagagcgttacatgggtctcccaattccagaagacaaccttgaccattacaggaactcaacagtcatgagcagagctgaacattttaaacaagttgagtacctccttattcatggaacggcagatgataatgttcactttcagcagtcagctcagatctccaaagccctggtggatgctggtgtggatttccaagcaatgtggtacacggatgaagaccacgggatcgctagcagcacagctcaccagcacatctattcccacatgagccatttcctccagcagtgcttctccttacactag

SEQ ID NO: 6 - protein, 760 aa, Mus musculus
MKTPWKVLLGLLGVAALVTIITVPIVLLSKDEAAADSRRTYSLADYLKSTFRVKSYSLWWVSDFEYLYKQENNILLLNAEHGNSSIFLENSTFESFGYHSVSPDRLFVLLEYNYVKQWRHSYTASYNIYDVNKRQLITEEKIPNNTQWITWSPEGHKLAYVWKNDIYVKVEPHLPSHRITSTGEENVIYNGITDWVYEEEVFGAYSALWWSPNNTFLAYAQFNDTGVPLIEYSFYSDESLQYPKTVWIPYPKAGAVNPTVKFFIVNIDSLSSSSSAAPIQIPAPASVARGDHYLCDVVWATEERISLQWLRRIQNYSVMAICDYDKINLTWNCPSEQQHVEMSTTGWVGRFRPAEPHFTSDGSSFYKIISDKDGYKHICHFPKDKKDCTFITKGAWEVISIEALTSDYLYYISNQYKEMPGGRNLYKIQLTDHTNVKCLSCDLNPERCQYYAVSFSKEAKYYQLGCWGPGLPLYTLHRSTDHKELRVLEDNSALDRMLQDVQMPSKKLDFIVLNETRFWYQMILPPHFDKSKKYPLLLDVYAGPCSQKADASFRLNWATYLASTENIIVASFDGRGSGYQGDKIMHAINRRLGTLEVEDQIEAARQFVKMGFVDSKRVAIWGWSYGGYVTSMVLGSGSGVFKCGIAVAPVSRWEYYDSVYTERYMGLPIPEDNLDHYRNSTVMSRAEHFKQVEYLLIHGTADDNVHFQQSAQISKALVDAGVDFQAMWYTDEDHGIASSTAHQHIYSHMSHFLQQCFSLH

SEQ ID NO: 7 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 8 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT